(19) United States
(12) Patent Application Publication
(10) Pub. No.: US 2008/0261890 A1
(43) Pub. Date: Oct. 23, 2008

(54) Use of neurotransmitters and neuropeptides for the treatment of dry eye diseases and related conditions

(76) Inventors: George W. Ousler, (US); Matthew J. Chapin, (US); Mark B. Abelson, (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/214,686

(22) Filed: Jun. 20, 2008

Related U.S. Application Data
(63) Continuation of application No. 11/295,165, filed on Dec. 6, 2005.
Application No. 11/295,165 is a continuation-in-part of application No. 11/087,096, filed on Mar. 21, 2005, now abandoned.
(60) Provisional application No. 60/555,031, filed on Mar. 19, 2004.

(51) Int. Cl. A61K 38/22; A61K 38/02; A61P 27/04; A61K 38/07

(52) U.S. Cl. 514/12; 514/2; 514/18

(57) ABSTRACT

The invention further features novel methods and compositions for treating and preventing dry eye by administration of neurotransmitters and/or neuropeptides, optionally in formulation with various other agents, as well as kits for the use of such novel formulations and methods.

### REFERENCE TO RELATED APPLICATIONS

This application is a continuation of U.S. Ser. No. 11/295,165, filed Dec. 6, 2005, which is a continuation-in-part of U.S. Ser. No. 11/087,096, filed Mar. 21, 2005, which in turn claims priority under 35 U.S.C. §119(e) to U.S. Ser. No. 60/555,031, filed Mar. 19, 2004, each of which are herein incorporated by reference in their entireties.

### BACKGROUND OF THE INVENTION

The human cornea is thought to be the most densely innervated tissue in the human body. Surface epithelial cells also contain several different varieties of receptors which are believed to be involved in the regulation of tear production and balance. Using methods ranging from corneal staining to confocal and electron microscopy, researchers have taken large steps toward an accurate portrayal of human corneal nerve structure. Tracing a path from the periphery of the cornea, nerve bundles initially penetrate parallel to the ocular surface and eventually weave radially outward, terminating as beaded fibers at the superficial epithelial cell layers. The vast majority are sensory nerves, while the concentrations of sympathetic and parasympathetic nerves are yet to be determined, though are believed to be minimal.

The interconnecting reflexive innervation of various contributory structures such as the cornea, conjunctiva, accessory lacrimal glands, meibomian glands, and the main lacrimal gland is thought to help to maintain the integrity of the ocular surface. The ocular surface is protected from the external environment by the tear film. The tear film consists of three separate layers: an inner mucin or glycolax layer, a middle aqueous, hydrated gel layer, and an outer lipid layer. Each layer plays an integral part in protecting the ocular surface. When a deficiency occurs in one of these layers, the tear film breaks down exposing the ocular surface to the drying effects of the external environment. Stimulation of the nerves beings a cascade of chemical steps that initiate an appropriate response to maintain the tear film and ocular surface. For example, in normal individuals, when corneal nerves are stimulated by environmental factors (e.g. low humidity, wind, contact lens, etc.) a reflex results in blinking and the secretion of supportive tear substances (e.g. proteins, mucins, lipids, and water) that can maintain and repair the ocular surface.

To date, 17 different neuropeptides and neurotransmitters have been discovered as the chemical agents for corneal innervation. Among these, the vasoactive intestinal peptide (VIP) has been shown to stimulate corneal epithelial cell production of nerve growth factors. It has also been demonstrated that VIP innervation exists in most of the major secretory glands in the eye. The signals conveyed are essential to maintain a healthy ocular surface, and any dysfunction can lead to neurotrophic keratitis and dry eye disease and related conditions via unregulated balance of tear film components or the inability to reflex tear. Other causes of desensitized nerves and other neural dysfunctions in the eye are herpetic keratitis, diabetes, prolonged contact lens wear, and advanced age.

Dry eye is an ocular disease affecting approximately 10-20% of the population. This disease progressively effects larger percentages of the population as it ages, with the majority of these patients being women. In addition, almost everyone experiences dry eye signs and/or symptoms from time to time under certain circumstances, such as prolonged visual tasking, working on a computer, being in a dry environment, etc.

In individuals suffering from dry eye, the reflex that results in blinking and the secretion of supportive tear substances is compromised. Signs and symptoms of dry eye include keratitis, conjunctival and corneal staining, redness, blurry visions, decreased tear film break-up time, decreased tear production, volume, and flow, increased conjunctival redness, excess debris in tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive, but it is a natural reflex response to the irritation and foreign body sensation caused by the dry eye. Some patients also experience ocular itching due to a combination of ocular allergy and dry eye symptoms.

There are many possible variables that also can influence a patient's symptoms of including levels of circulating hormones, various autoimmune diseases (e.g. Sjorgren's syndrome and systemic lupus erythematosus), ocular surgeries including PRK or LASIK, many medications, environmental conditions, visual tasking such as computer use, ocular fatigue, contact lens wear, and mechanical influences such as corneal sensitivity, partial lid closure, surface irregularities (e.g. pterygium), and lid irregularities (e.g. ptosis, entropion/ectropion, pinguecula). Environments with low humidity can exacerbate or cause dry eye symptoms, such as sitting in a car with the defroster on or living in a dry climate zone. In addition, visual tasking can also exacerbate symptoms. Tasks that can greatly influence symptoms include watching TV or using a computer for long periods of time where the blink rate is decreased. When the blink rate is decreased, the tear film is not replaced on the ocular surface often enough leaving the ocular surface exposed to the external environment.

It has also been shown that certain diseases which alter the autonomic-nervous-system function can also result in the signs and symptoms of dry eye. For example, Riley Day Syndrome or familial dysautomonia is characterized by a decreased level of the synthesis of dopamine and symptoms of sensory disturbances, occurring primarily in Ashkenazi Jewish children and appears to be inherited in an autosomal recessive manner. Clinical studies have shown that this population of patients presents with decreased blink rate and exacerbated signs and symptoms of dry eye.

An increasingly prevalent cause of neurotrophic keratitis is keratorefractive surgery such as radial keratotomy (PRK), photorefractive keratectomy and laser assisted in situ keratomileusis (LASIK), which damage stromal nerves and the corneal subbasal plexus, where the corneal nerve endings are severed in the course of surgery. It has been found that ocular dryness and irritation occur in over one half of LASIK patients. LASIK surgery causes dry eye due to the severing of nerves that run to the ocular surface leading to decreased corneal sensitivity. Corneal sensitivity is linked with the lacrimal gland functioning; decreased corneal sensation leads to decreased secretions from the lacrimal gland causing dry eye.

Recent cosmetic trends indicate that more people are wearing contact lenses for longer periods of time and more people are having refractive surgery, and thus dry eye disease is likely to affect greater numbers of people in the future. Further, the aging population of baby boomers indicates that dry eye diseases will be a significant concern in the future.

Therefore, a treatment that can assist in maintaining the neural regulation of the ocular surface and minimize the signs and symptoms of dry eye disease and related conditions is desirable.

### SUMMARY OF THE INVENTION

The invention features novel pharmaceutical formulations of neurotransmitters and/or neuropeptides, optionally in combination with various other agents (such as a tear substitute), for the treatment of dry eye. The invention also features novel methods of treating and preventing dry eye by administration of at least one neurotransmitter and/or neuropeptide. Further, the invention features kits for the shipping, storage or use of the formulations, as well the practice of the methods. Other features and advantages of the invention will become apparent from the following detailed description and claims.

### DETAILED DESCRIPTION OF THE INVENTION

### 1. General

In dry eye, one or more than one of several mechanisms for maintaining the integrity of the ocular surface is not functioning properly or is not present, such that the ocular surface is compromised. In particular, dry eye may result if certain neurotransmitter(s) is/are not present at sufficient levels to induce the neural signal transmission in the cornea which is needed for sensation to drive tearing (production of aqueous and/or mucins and/or lipids) and blinking. Both tearing (and the quality of tears) and/or blinking are critical components of maintaining a healthy ocular surface. Reductions in these factors can contribute to signs and/or symptoms that may result in dry eye.

### 2. Definitions

For convenience, before further description of the present invention, certain terms employed in the specification, examples, and appended claims are collected here. These definitions should be read in light of the remainder of the disclosure and understood as by a person of skill in the art.

The articles “a” and “an” are used herein to refer to one or to more than one (i.e., to at least one) of the grammatical object of the article.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “aqueous” typically denotes an aqueous composition wherein the carrier is to an extent of >50%, more preferably >75% and in particular >90% by weight water.

As used herein, the term “antiallergenic agent” refers to a molecule or composition that treats ocular allergy or reduces a symptom of ocular allergy. Examples of antiallergenic agents include, but are not limited to, “antihistamines” or drugs which block histamine from binding to the histamine receptors, “mast cell stabilizers” or drugs that block the release of histamine and other substances from the mast cell, “drugs with multiple modes of action” or drugs that are antiallergenic agents having multiple modes of action (e.g. drugs that are antihistamines and mast cell stabilizers, drugs with antihistamine, mast cell stabilizing and anti-inflammatory activity, etc.), and nonsteroidal anti-inflammatory drugs or “NSAIDs.”

The term “dry eye” as used herein means any disease or disorder or condition which results in an adverse effect on the quality of the tear film that lubricates the eyes. The disease or disorder may be of the eye itself, or of another part of the body, so long as it results in an adverse effect on the quality of the tear film that lubricates the eyes. For example, “dry eye” as used herein includes dry eye disorder, Riley Day Syndrome and keratitis, as well as dry eye caused by other conditions, factors and phenomena such as diabetes, prolonged contact lens wear, advanced age, circulating hormones, various autoimmune diseases (e.g. Sjorgren's syndrome and systemic lupus erythematosus), ocular surgeries including PRK or LASIK, many medications, environmental conditions, visual tasking such as computer use, ocular fatigue, mechanical influences such as corneal sensitivity, partial lid closure, surface irregularities (e.g. pterygium), and lid irregularities (e.g. ptosis, entropion/ectropion, pinguecula).

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of dry eye, or prevent or treat dry eye. The effective amount may vary depending on such factors as the disease or condition being treated, the particular composition being administered, or the severity of the disease or condition. One of skill in the art may empirically determine the effective amount of a particular agent without necessitating undue experimentation.

The term “hormone” refers to any molecule that is produced by a specific cell or tissue and causes a change or activity in a cell or tissue located elsewhere in an organism.

The term “neurotransmitter” as used herein means any molecule or compound, which is released from the axon of one neuron and binds to a specific site in the dendrite of an adjacent neuron, thus triggering a nerve impulse. A neurotransmitter may be, for example, a small molecule, a peptide, an amino acid, a hormone, a protein, a vitamin, or a free radical.

The term “neuropeptide” as used herein means a peptide with a direct synaptic effect (i.e., a peptide that is a neurotransmitter) and/or an indirect effect on synaptic transmission. Neuropeptides may be released from neurons or from non-neuronal cells, and may also act as hormones.

The term “ocular allergy” as used herein refers to any allergic disease of the eye. Examples of such ocular allergies include but are not limited to seasonal/perennial allergic conjunctivitis, vernal keratoconjunctivitis, giant papillary conjunctivitis, perennial allergic conjunctivitis and atopic keratoconjunctivitis. The signs and symptoms of ocular allergies include chemosis, eye itching, redness and swelling.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “polypeptide”, and the terms “protein” and “peptide” which are used interchangeably herein, refers to a polymer of amino acids. Exemplary polypeptides include gene products, naturally-occurring peptides and proteins, homologs, orthologs, paralogs, fragments, and other equivalents, variants and analogs of the foregoing.

The term “preventing,” when used in relation to a condition, such as dry eye, is art-recognized, and refers to administration of a composition which reduces the frequency of, or delays the onset of, symptoms of a medical condition in a subject relative to a subject which does not receive the composition.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

As used herein, the term “tear substitute” refers to molecules or compositions which lubricate, “wet,” approximate the consistency of endogenous tears, aid in natural tear build-up, or otherwise provide temporary relief of dry eye symptoms and conditions upon ocular administration.

The term “treating” is an art-recognized term which refers to curing as well as ameliorating at least one symptom of any condition or disease.

“Vasoconstrictors” are drugs that actively constrict blood vessels.

### 3. Pharmaceutical Compositions

In one aspect, the invention features novel pharmaceutical compositions comprising an effective amount of at least one neurotransmitter or neuropeptide in a pharmaceutically acceptable carrier for the treatment and prevention of dry eye.

In certain embodiments, the composition comprises at least one neurotransmitter. In order to be classified as a neurotransmitter, a substance must meet the following conditions: a) synaptic vesicles in axon terminals of the pre-synaptic neuron must contain the substance and release it in response to a stimulation of sufficient magnitude such that the signal is induced in the postsynaptic cell; b) direct application of the substance to the postsynaptic neuron must induce the same response as stimulation of the presynaptic neuron. Exemplary neurotransmitters include, but are not limited to, acetylcholine, ATP, glycine, glutamate, dopamine, norepinephrine, epinephrine, octopamine, serotonin (5-hydroxytryptamine), beta-alanine, histamine, gamma-aminobutyric acid (GABA), taurine, aspartate and nitric oxide. Accordingly, neurotransmitters may be small molecules, peptides, amino acids, hormones, proteins, vitamins or free radicals.

In other embodiments, the composition comprises at least one neurokinase agent, e.g. an agonist, antagonist or other modulator of neurokinase activity. A “kinase” is an enzyme that catalyzes the transfer of phosphate groups from a high-energy phosphate-containing molecule (as ATP or ADP) to a substrate. Accordingly, a “neurokinase” is a kinase that catalyzes the transfer of phosphate groups between molecules involved in neurotransmission, such as β-adrenergic receptors and 5-HT receptors.

In other embodiments, the composition comprises at least one neuropeptide. In addition to “classic” neurotransmitters such as those listed above, there is a growing list of peptide molecules produced and released in the nervous system that act as neurotransmitters or which influence synaptic transmission. These neurotransmitters are also be known in the art as “neurosecretory substances.” Exemplary neuropeptides include, but are not limited to, hypothalamic hormones such as oxytocin (9 amino acid residues, “a.a.r.”) and vasopressin (9 a.a.r.); hypothalamic releasing and inhibiting hormones such as corticotropin releasing hormone (CRH) (41 a.a.r.), growth hormone releasing hormone (GHRH) (44 a.a.r.), luteinizing hormone releasing hormone (LHRH) (10 a.a.r.), somatostatin growth hormone release inhibiting hormone (14 a.a.r. plus several forms) and thyrotropin releasing hormone (TRH) (3 a.a.r.); tachykinins such as neurokinin a (substance K) (10 a.a.r.), neurokinin b (10 a.a.r.), neuropeptide K (36 a.a.r.) and substance P (11 a.a.r.); opioid peptides such as b-endorphin (30 a.a.r.), dynorphin (17 a.a.r. and other forms) and met- and leu-enkephalin (5 a.a.r.); NPY and related peptides such as neuropeptide tyrosine (NPY) (36 a.a.r.), pancreatic polypeptide (36 a.a.r) and peptide tyrosine-tyrosine (PYY) (36 a.a.r.); VIP-glucagon family members such as glucogen-like peptide-1 (GLP-1) (29 a.a.r.), peptide histidine isoleucine (PHI) (27 a.a.r.), pituitary adenylate cyclase activating peptide (PACAP) (27 or 38 a.a.r.) and vasoactive intestinal polypeptide (VIP) (28 a.a.r.); as well as many other peptides such as brain natriuretic peptide (32 a.a.r.), calcitonin gene-related peptide (CGRP) (a- and b-form) (37 a.a.r.), cholecystokinin (CCK) (8 a.a.r. and other forms), galanin (29 or 30 a.a.r.), islet amyloid polypeptide (LAPP) or amylin (37 a.a.r), melanin concentrating hormone (MCH) (19 a.a.r.), melanocortins (ACTH, a-MSH and others), neuropeptide FF (F8Fa) (8 a.a.r), neurotensin (13 a.a.r.), parathyroid hormone related protein (34 or 37 a.a.r.), Agouti gene-related protein (AGRP) (13 a.a.r.), cocaine and amphetamine regulated transcript (CART)/peptide, endomorphin-1 and -2 (both 4 a.a.r.), 5-HT-moduline (4 a.a.r.), hypocretins/orexins (29 or 39 a.a.r.), nociceptin/orphanin FQ (17 a.a.r.), nocistatin (17 a.a.r.), prolactin releasing peptide (20 or 31 a.a.r.), secretoneurin (33 a.a.r.) and urocortin (40 a.a.r.; 45% sequence identity with CRH).

In other embodiments, the pharmaceutical compositions may comprise more than one neurotransmitter or neuropeptide. For example, a pharmaceutical composition may comprise two neurotransmitters, or two neuropeptides, or a neurotransmitter and a neuropeptide.

The pharmaceutical compositions described above may additionally comprise one or more additional active ingredients, including, but not limited to, tear substitutes, antiallergenic agents and vasoconstrictors. Such compositions may be used, for example, to treat or prevent not only dry eye but an underlying or concurrent disorder or disease such as ocular allergy or to treat or prevent symptoms accompanying dry eye. For example, a pharmaceutical composition may comprise a neurotransmitter or neuropeptide, and a tear substitute. Or, it may comprise a neurotransmitter or neuropeptide, and an antiallergenic agent, and optionally a tear substitute.

Exemplary uses of various agents in treating ocular allergy and compositions thereof are described in U.S. patent application Ser. No. 10/762,201 filed Mar. 20, 2004 and United States patent application filed Mar. 3, 2005, serial number not yet assigned, which claims priority to U.S. Provisional Patent Application 60/549,703, filed Mar. 3, 2004, all of which applications are incorporated by reference in their entireties. The dosages and combinations of agents described therein, for example, may be combined with the presently described neurotransmitter/neuropeptide formulations.

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Teargen II®, Tears Naturale®, Tears Naturale II®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

Exemplary antihistamines include, but are not limited to, pheniramine, emedastine difumarate and levocabastine. In other embodiments, the invention features pharmaceutical compositions comprising an effective amount of a mast cell stabilizer and a tear substitute. Exemplary mast cell stabilizers include, but are not limited to, nedocromil, lodoxamide, cromolyn, and cromolyn sodium. Exemplary drugs with multiple modes of action include, but are not limited to, azelastine, epinastine, olopatadine and ketotifen fumarate.

Exemplary vasoconstrictors include, but are not limited to, naphazoline, antolazine, tetrahydrozoline and oxymetazoline.

The antiallergenic agents and other active ingredients of the pharmaceutical compositions may be in the form of a pharmaceutically acceptable salt.

### 4. Formulations of Pharmaceutical Compositions

Methods of formulating and formulations of the above pharmaceutical compositions are also included in the invention. The effective amount of neurotransmitter and/or neuropeptide in a formulation will depend on absorption, inactivation, and excretion rates of the drug as well as the delivery rate of the neurotransmitter and/or neuropeptide from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

For example, each of a neurotransmitter and/or neuropeptide may be present in the composition at a dose in the range of about 0.001 to about 10.0%. For example, the effective amount of each of a neurotransmitter or neuropeptide may be in the range of about 0.001 to about 0.01%, of about 0.01 to about 0.100%, of about 0.100 to about 1.0%, or of about 1.00 to about 10.00%.

Preferably, the pharmaceutical compositions according to the present invention will be formulated as solutions, suspensions and other dosage forms for topical ophthalmic administration in a pharmaceutically acceptable carrier, adjuvant, or vehicle. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the topical formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100,000 times the concentration of the active ingredient.

Additional ingredients that may be included in the formulations include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N—(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

In other embodiments, the pharmaceutical compositions according to the present invention will be formulated for other types of administration, such as oral, parenteral, inhalation spray, rectal, nasal, buccal, vaginal, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques. Methods of formulating pharmaceutical compositions for such forms of administration are well-known to one of skill in the art.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a molecule thereof as an active ingredient. Compositions of the present invention may also be administered as a bolus, electuary, or paste.

In solid dosage forms for oral administration (capsules, tablets, pills, dragees, powders, granules and the like), the particle is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium molecules; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the supplement or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in p articular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a particle of the present invention with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

### 5. Methods of Treating and Preventing Dry Eye

The administration of a treatment comprising a neurotransmitter and/or neuropeptide, administered alone or in combination in another agents or other neurotransmitters and/or neuropeptides, may be used to treat or prevent dry eye in a subject. Administration of a neurotransmitter and/or neuropeptide to a subject may help stimulate the neural pathway that otherwise is altered by the disorder, disease, factor or phenomenon causing dry eye in the subject and positively affect the tear film and/or blinking to treat or prevent dry eye in the subject.

The dosage will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages may be readily determined by techniques known to those of skill in the art or as taught herein.

An effective dose or amount, and any possible effects on the timing of administration, may need to be identified for any particular formulation or method of the present invention. This may be accomplished by routine experiment as described herein. The effectiveness of any formulation and method of treatment or prevention may be assessed by administering the formulation and assessing the effect of the administration by measuring one or more indices associated with the efficacy of the antiallergenic agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. Guidelines may be used to optimize the treatment, e.g., determining the optimum time and/or amount of administration, which will require no more than routine experimentation consisting of monitoring the subject and adjusting the dosage and/or timing.

The combined use of several neurotransmitters and/or neuropeptides formulated into the compositions of the present invention may reduce the required dosage for any individual component because the onset and duration of effect of the different components may be complimentary. In such combined therapy, the different agents may be delivered together or separately, and simultaneously or at different times within the day.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of one week from the onset of therapy, and subsequent reevaluations occurring every one to two weeks during therapy and then every month thereafter. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Toxicity and therapeutic efficacy may be determined by standard pharmaceutical procedures in cell cultures or experimental animals, e.g., for determining the LD₅₀ and the ED₅₀. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

### 6. Packaging

The topical formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### 7. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

### REFERENCES

All publications and patents mentioned herein, including those listed below, are hereby incorporated by reference in their entireties as if each individual publication or patent was specifically and individually indicated to be incorporated by reference. In case of conflict, the present application, including any definitions herein, will control.
Marfurt C. Nervous control of the cornea. Innervation of the Eye. Gordon and Breach Science, London. 1997.
Stern M, Beuerman R W. A unified theory of the role of the ocular surface in dry eye. Lacrimal Gland, Tear Film, and Dry Eye Syndromes 2. 1998. 643-651.
Muller U, Marfurt C F, Kruse F, Tervo T M T. Corneal nerves: Structure, contents and function. Experimental Eye Research. 76. 2003. 521-42
Wilson S E. Analysis of the keratocyte apoptosis, keratocyte proliferation, and myofibroblast transformation responses after photorefractive keratectomy and laser in situ keratomileusis. Trans Am Opthalmol Soc. 2002; 100: 411-33.
Matsuo T, Tsuchida Y, Morimoto N. “Trehalose eye drops in the treatment of dry eye syndrome.” Opthalmology, 2002. Vol. 109. Pages 2024-2029.
Watanabe H. “Significance of mucin on the ocular surface.” Cornea 2002. Vol. 21. Pages S17-S22.
Gipson I K and Inatomi T. “Cellular origin of mucins of the ocular surface tear film.” In Lacrimal Gland, Tear Film and Dry Eye Syndromes 2. Edited by Sullivan et al. Plenum Press, NY. 1998.
Battat L, Macri A, Dursun D, Pflugfleder S C. “Effects of laser in situ keratomileusis on tear production, clearance, and the ocular surface.” American Academy of Opthalmology, 2001. Pages 1230-1235.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

We claim:

1. A pharmaceutical composition formulated for topical ophthalmic use comprising an effective amount of at least one neurotransmitter or at least one neuropeptide in a pharmaceutically acceptable carrier.

2. The pharmaceutical composition of claim 1, wherein the composition comprises at least one neurotransmitter and wherein the neurotransmitter is selected from the group consisting of: acetylcholine, ATP, glycine, glutamate, dopamine, norepinephrine, epinephrine, octopamine, serotonin (5-hydroxytryptamine), beta-alanine, histamine, gamma-aminobutyric acid (GABA), taurine and aspartate.

3. The pharmaceutical composition of claim 1, wherein the composition comprises at least one neuropeptide and wherein the neuropeptide is selected from the group consisting of: hypothalamic hormones, hypothalamic releasing and inhibiting hormones, opioid peptides, NPY and related peptides, VIP-glucagon family members, brain natriuretic peptide, calcitonin gene-related peptide (CGRP), cholecystokinin (CCK), galanin, islet amyloid polypeptide (IAPP) or amylin, melanin concentrating hormone (MCH), melanocortins (ACTH, a-MSH and others), neuropeptide FF (F8Fa), neurotensin, parathyroid hormone related protein, Agouti gene-related protein (AGRP), cocaine and amphetamine regulated transcript (CART)/peptide, endomorphin-1 and -2, 5-HT-moduline, hypocretins/orexins, nociceptin/orphanin FQ, nocistatin, prolactin releasing peptide, secretoneurin and urocortin.

4. The pharmaceutical composition of claim 1, wherein the composition comprises more than one neurotransmitter or neuropeptide.

5. The pharmaceutical composition of claim 1, wherein the pharmaceutical composition further comprises a tear substitute.

6. The pharmaceutical composition of claim 5, wherein the tear substitute contains hydroxypropylmethylcellulose.

7. The pharmaceutical composition of claim 1, wherein the pharmaceutical composition further comprises at least one antiallergenic agent.

8. The pharmaceutical composition of claim 7, wherein the antiallergenic agent is selected from the group consisting of: an antihistamine, a mast cell stabilizer, a drug with multiple modes of action and a NSAID.

9. The pharmaceutical composition of claim 8, wherein the antiallergenic agent is a drug with multiple modes of action.

10. The pharmaceutical composition of claim 9, wherein the drug with multiple modes of action is ketotifen fumarate.

11. The pharmaceutical composition of claim 1, wherein the composition further comprises at least one antiallergenic agent and a tear substitute.

12. A method of treating dry eye in a subject comprising: administering to the eye surface of the subject a pharmaceutical composition formulated for topical ophthalmic use comprising an effective amount of at least one neurotransmitter or at least one neuropeptide in a pharmaceutically acceptable carrier.

13. The method of claim 12, wherein the composition comprises at least one neurotransmitter and wherein the neurotransmitter is selected from the group consisting of: acetylcholine, ATP, glycine, glutamate, dopamine, norepinephrine, epinephrine, octopamine, serotonin (5-hydroxytryptamine), beta-alanine, histamine, gamma-aminobutyric acid (GABA), taurine and aspartate.

14. The method of claim 12, wherein the composition comprises at least one neuropeptide and wherein the neuropeptide is selected from the group consisting of: hypothalamic hormones, hypothalamic releasing and inhibiting hormones, opioid peptides, NPY and related peptides, VIP-glucagon family members, brain natriuretic peptide, calcitonin gene-related peptide (CGRP), cholecystokinin (CCK), galanin, islet amyloid polypeptide (IAPP) or amylin, melanin concentrating hormone (MCH), melanocortins (ACTH, a-MSH and others), neuropeptide FF (F8Fa), neurotensin, parathyroid hormone related protein, Agouti gene-related protein (AGRP), cocaine and amphetamine regulated transcript (CART)/peptide, endomorphin-1 and -2, 5-HT-moduline, hypocretins/orexins, nociceptin/orphanin FQ, nocistatin, prolactin releasing peptide, secretoneurin and urocortin.

15. The method of claim 12, wherein the composition comprises more than one neurotransmitter or neuropeptide.

16. The method of claim 12, further comprising administering to the eye surface of the subject a tear substitute.

17. The method of claim 12, further comprising administering to the eye surface of the subject at least one antiallergenic agent.

18. The method of claim 12, further comprising administering to the eye surface of the subject at least one antiallergenic agent and a tear substitute.

19. A kit comprising a pharmaceutical composition formulated for topical ophthalmic use comprising an effective amount of at least one neurotransmitter in a pharmaceutically acceptable carrier.

20. The kit of claim 19, further comprising instructions for use.
